(19) United States
(12) Patent Application Publication
(10) Pub. No.: US 2008/0249386 A1
(43) Pub. Date: Oct. 9, 2008

(54) Systems, Methods, and Computer Program Product for Improved Management of Medical Procedures for Patients on Medical Protocols

(75) Inventors: Brian J. Besterman, South Salem, NY (US); Michael R. Marvin, Lousiville, KY (US); James A. Jorasch, New York, NY (US)

Correspondence Address: PRIEST & GOLDSTEIN PLLC, 5015 SOUTHPARK DRIVE, SUITE 230, DURHAM, NC, 27713-7736, US

(73) Assignee: Pronia Medical Systems, LLC, Louisville, KY (US)

(21) Appl. No.: 12/061,856

(22) Filed: Apr. 3, 2008

Related U.S. Application Data
(60) Provisional application No. 60/921,764, filed on Apr. 4, 2007.

(51) Int. Cl. A61B 5/145; G06Q 50/00; A61M 5/14

(52) U.S. Cl. 600/365; 705/3; 604/66

(57) ABSTRACT

Techniques are described for improved monitoring and control of a patient's physiologic status, such as blood glucose control according to a protocol for use in a patient care unit, such as an intensive care unit. Instructions to medical staff are adapted to factor in variations in responses to protocol recommendations. For example, a patient's physiological data as a result of a blood analysis is submitted as input to a program. An instruction for a medical procedure for the patient in response to the physiological data is automatically provided by the program. A confirmation response that the medical procedure has been accomplished and results of following the medical procedure are requested by the program. The time when the medical procedure was accomplished and the results of following the medical procedure are evaluated by the program to determine whether to adapt a subsequent instruction.

### CROSS REFERENCE TO RELATED APPLICATION

The present application claims the benefit of U.S. Provisional Patent Application No. 60/921,764 entitled “Apparatus, Systems and Methods for Improved Patient Glucose Control”, filed on Apr. 4, 2007 which is hereby incorporated by reference in its entirety.

### FIELD OF THE INVENTION

The present invention relates generally to systems, methods, and computer program products for improved management of medical procedures for patients on medical protocols, and more particularly to advantageous techniques and models for improved management of medical procedures for patients on glucose control protocols.

### BACKGROUND OF INVENTION

Medical studies have shown that maintaining blood glucose levels of patients in a hospital intensive care unit (ICU) within a narrow range can have many benefits, such as shorter hospital stays, fewer complications, and lower costs to the patient and hospital. However, blood glucose levels can be difficult to control in patients with underlying illnesses, or who have recently undergone major surgery. Some common methods of glucose control such as sliding insulin scales, react or respond to measured high glucose levels instead of preventing them in the first place. Also, patients on these common methods often do not experience the benefits of other methods of control that prevent the high glucose levels.

In the past few years, glucose control protocols have started to be used that aim to rapidly bring blood glucose levels into a narrow euglycemic range, and maintain blood glucose levels within this range in a safe manner. One such protocol is the Yale Infusion Protocol, which was developed at the Yale Medical Center, and consists of a set of written instructions that dictate insulin drip rates based on a patient's blood glucose readings. The instructions include calculations that are usually performed by hand or with a simple calculator. The instructions also include steps that must occur at particular time intervals. Unfortunately, these protocols are difficult to administer without errors in both the calculations required and the timing of the steps. Many other problems arise in the use of these manual control protocols. For example, since the manual protocols increase the work load of nursing staff or provide little or no flexibility in their instructions, the protocols are many times not followed as recommended. Studies have shown that about 50% of all patients on several of these prior protocols have experienced at least one protocol error during a course of treatment. Such errors make it difficult to conduct rigorous studies of protocol efficacy. Large scale studies of these protocols are being considered, but will be difficult to perform without some ability to prevent the many errors that typically occur.

### SUMMARY OF INVENTION

Among its several aspects, the present invention recognizes that there is a need to improve how patients are managed on medical protocols in order to reduce the burden of following manual protocols, decrease errors, account for the unpredictable and busy environment of an ICU, account for medical staff with different roles and responsibilities, allow for medical judgment, and provide escalating alerts, all with the ultimate goal of improving the care of patients on these protocols. To such ends, systems, methods, and computer program products for managing medical procedures for patients on medical protocols are described in the present invention. An embodiment of the present invention includes a method for adapting management of medical procedures for a patient assigned to a medical protocol. A report of a patient's monitored physiological data and confirmed medical procedures is periodically received at a management process from a patient's monitoring station. An instruction for management of the medical procedures for the patient is adapted in response to an evaluation of the patient's periodic reports. The adapted instruction is sent from the management process to the patient's monitoring station or to selected other monitoring stations based on the evaluation.

An embodiment of the present invention addresses a system for monitoring a patient and providing instructions to medical staff for controlling one or more of a patient's physiological parameters. A computing device is utilized for running a program to monitor a patient and generate instructions to control a physiological parameter of the patient. A user terminal is associated with the patient for monitoring and communicating instructions to the medical staff, and for receiving the one or more of the patient's physiological parameters from the medical staff which are then sent to the computing device. A blood sampling and analysis means provides an analysis of a blood sample taken from the patient, the analysis is in a suitable form to be submitted to the computing device. A medication administering device adjustable for dosage is utilized for controlling one or more doses of medication related to the patient's physiological parameters.

Another embodiment of the present invention addresses a computer-readable medium storing a computer program which causes a computer system to perform a method for adapting management of medical procedures for a patient assigned to a medical protocol. A report of a patient's monitored physiological data and confirmed medical procedures is periodically received at a management process from a patient's monitoring station. An instruction for management of the medical procedures for the patient is adapted in response to an evaluation of the patient's periodic reports. The adapted instruction is sent from the management process to the patient's monitoring station or to selected other monitoring stations based on the evaluation.

A more complete understanding of the present invention, as well as other features and advantages of the invention, will be apparent from the following detailed description and the accompanying drawings.

### BRIEF DESCRIPTION OF DRAWINGS

FIG. 1A illustrates an exemplary monitoring and control system for managing treatment of patients in a medical facility such as a hospital in accordance with an embodiment of the present invention;

FIG. 1B illustrates an exemplary remote monitoring and control system utilizing electronic communication with a medical facility in accordance with an embodiment of the present invention;

FIG. 1C illustrates an exemplary handheld system based monitoring and control system in accordance with an embodiment of the present invention;

FIG. 2 illustrates an exemplary prestart process in accordance with the present invention;

FIG. 3 illustrates an exemplary separation of concerns decision in an insulin rate adjustment process in accordance with the present invention;

FIG. 4 illustrates an exemplary main protocol process in accordance with the present invention;

FIG. 5 illustrates an exemplary check interval process in accordance with the present invention;

FIG. 6A illustrates an exemplary hypoglycemic blood glucose (BG) less than a warning level process in accordance with the present invention;

FIG. 6B illustrates an exemplary hypoglycemic sub-process in accordance with the present invention;

FIG. 6C illustrates an exemplary timer path process in accordance with the present invention;

FIG. 6D illustrates an exemplary hypoglycemic sub-process in accordance with the present invention;

FIG. 7A illustrates a station alert process in accordance with the present invention; and

FIG. 7B illustrates an alert management process in accordance with the present invention.

### DETAILED DESCRIPTION

The present invention will now be described more fully with reference to the accompanying drawings, in which several embodiments and various aspects of the invention are shown. This invention may, however, be embodied in various forms and should not be construed as being limited to the embodiments set forth herein. Rather, these embodiments are provided so that this disclosure will be thorough and complete and will fully convey the scope of the invention to those skilled in the art.

It will be appreciated that the present disclosures may be embodied as methods, systems, or computer program products. Accordingly, the present inventive concepts disclosed herein may take the form of a hardware embodiment, a software embodiment or an embodiment combining software and hardware aspects. Furthermore, the present inventive concepts disclosed herein may take the form of a computer program product on a computer-usable storage medium having computer-usable program code embodied in the medium. Any suitable computer readable medium may be utilized including hard disks, CD-ROMs, optical storage devices, flash memories, or magnetic storage devices.

Computer program code or software programs that are operated upon or for carrying out operations according to the teachings of the invention may be written in a high level programming language such as C, C++, JAVA® Smalltalk, JavaScript®, Visual Basic®, TSQL, Perl, use of .NET™ Framework, Visual Studio® or in various other programming languages. Software programs may also be written directly in a native assembler language for a target processor. A native assembler program uses instruction mnemonic representations of machine level binary instructions. Program code or computer readable medium as used herein refers to code whose format is understandable by a processor. Software embodiments of the disclosure do not depend upon their implementation with a particular programming language.

The methods described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. A computer-readable storage medium may be coupled to the processor through local connections such that the processor can read information from, and write information to, the storage medium or through network connections such that the processor can download information from or upload information to the storage medium. In the alternative, the storage medium may be integral to the processor.

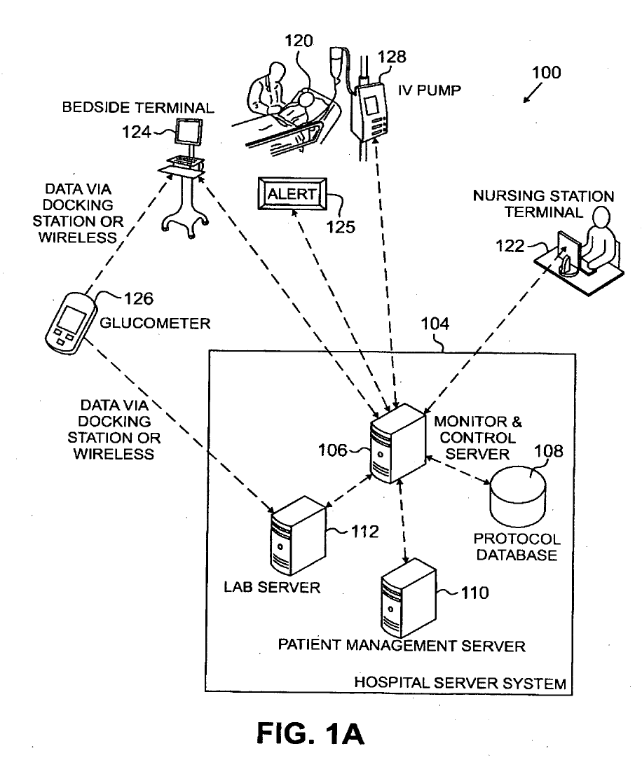

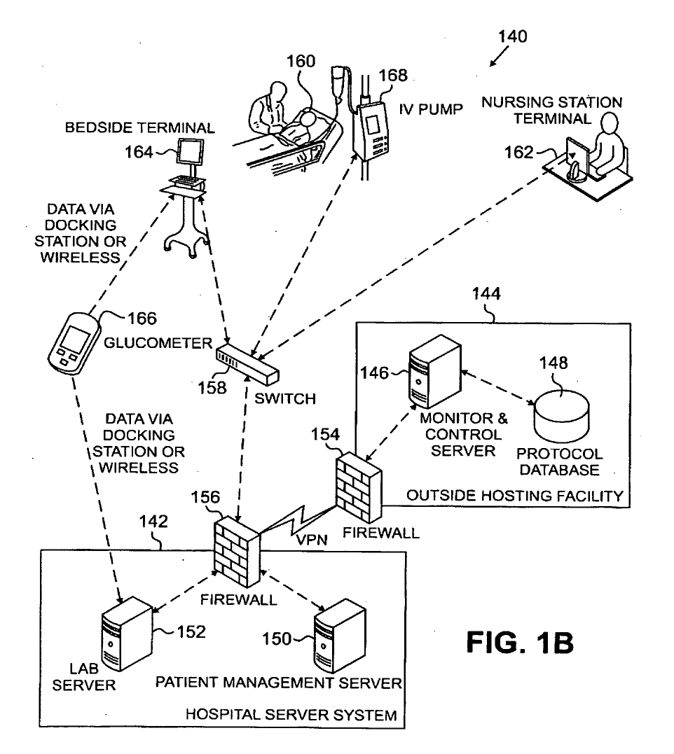

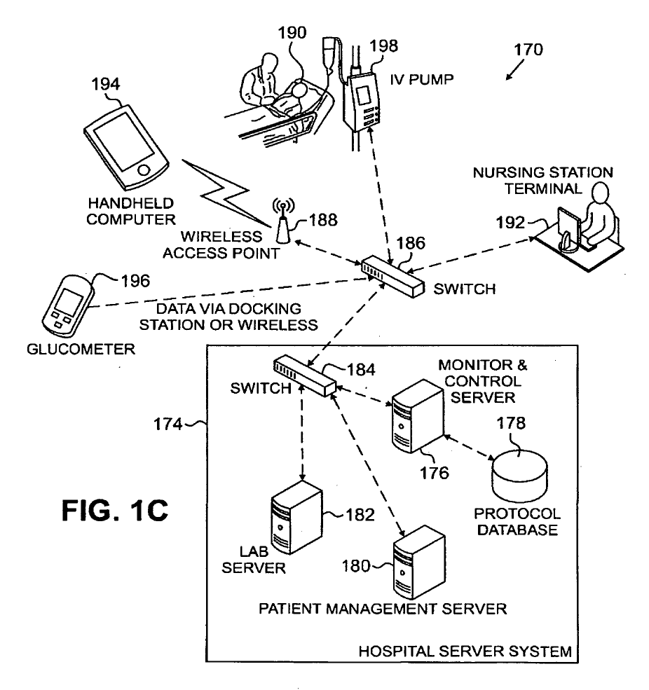

FIG. 1A illustrates a monitoring and control system 100 for managing treatment of patients in a medical facility such as a hospital in accordance with an embodiment of the present invention. The monitoring and control system 100 may suitably include a centralized server system 104 employing, for example, a monitor and control server 106, a database 108, a patient management server 110, and a lab server 112. Each of the servers, 106, 110, and 112 may include a processor complex having one or more processors, having internal program storage and local user controls such as a monitor, a keyboard, a mouse, a printer, and may include other input or output devices, such as an external file storage device and communication interfaces. The monitor and control server 106 may store programs such as a program implementation of a monitor and control process of the present invention or have access to such programs through electronic media, such as may be downloaded over the Internet from an external server, accessed through a universal serial bus (USB) port from flash memory, accessed from disk media of various types, or the like.

The server 106 has access to the database 108 which may be accessed by software programs operating from server 106, for example. The database 108 may store the patients' medical data, as well as all data related to inputs to and outputs from the system, and a plurality of medical protocols that have been adapted for use as described herein and in accordance with the present invention. It is noted that depending on the size of an installation, the functions of the monitor and control server 106, the database 108, the patient management server 110, and the lab server 112 may be combined in a single server running separate program threads for each function.

The monitoring and control system 100 may also suitably include one or more nursing station terminals 122, a patient bedside terminal 124 associated with each assigned patient 120, an alert device 125, a blood testing device for reading blood glucose levels, such as a glucometer 126, and an intravenous (IV) pump 128. The patient bedside terminals and nursing station terminals may collectively be called user terminals. Each of these devices may be connected directly to the patient monitor and control server 106 or indirectly connected to it over a network, such as a local cabled intra-net, wireless intra-net, the Internet, or the like. The nursing station terminals 122 may comprise, for example, a personal computer, a laptop computer, or the like. The patient bedside terminal 124 may comprise a personal computer equipped with interfaces to support local monitoring of a patient's cardiac rhythm, blood pressure, and other physiological data that may be taken both automatically or manually under professional supervision. The nursing stations terminals 122, bedside terminal 124, and IV pump 128 may issue audible and visual alerts as may be determined locally or under command, for example, from the monitor and control server 106. The patient bedside terminal 124 and nursing station terminals 122 may also have access to electronic medical records as may be accessed from the patient management server 110, lab information as may be accessed from the lab server 112, nursing care information and the like.

For example, a user terminal may support the presentation of graphs of insulin rate and glucose levels versus time. The user terminals may also support the viewing and printing of a history of recommendations made and responses to those recommendations as well as any other medical procedures taken in the care of the patient. For example, a first set of medical procedures may include determining a patient's blood glucose level and a second set of medical procedures may include changing an insulin infusion rate. The user terminal may also have access to historical data of patients under the medical staffs care who were monitored in the past. These terminals may further provide administrative support, such as adding a new patient, new users, change user permissions and passwords, and the like. In addition, a user terminal may allow access to the Internet and entertainment programs.

The blood testing device, such as glucometer 126, is utilized to measure blood glucose (BG) levels based on a small drop of blood taken from the patient, which is presently manually taken with fingersticks. The drop of blood is dabbed onto a test strip which is placed in the glucometer and the blood glucose (BG) reading is read on a display. A number of glucometers may allow connection via a docking station or through a wireless coupling to upload their data, for example, to the patient bedside terminal 124, the nursing station terminals 122, or to the lab server 112. It is anticipated that a closed-loop system may be provided which utilizes an implantable glucose sensor that responds to commands or periodically sends BG readings directly to a user terminal or the lab server. The IV pump 128 is utilized to control the rates that IV fluids or IV medications are given to a patient and may be electronically set or manually set under professional supervision. The IV pump 128 may have displays, key entry means through a key pad or on-screen keys or both. It will be recognized that many of a user terminal's functions, such as may be included in the patient bedside terminal 124 or nursing station terminals 122, could be implemented in an appropriately equipped IV pump.

FIG. 1B illustrates an exemplary remote monitoring and control system 140 utilizing electronic communication with a medical facility in accordance with an embodiment of the present invention. The remote monitoring and control system 140 may be organized with one or more hospital server systems, such as the hospital server system 142 coupled via the Internet over, for example, a virtual private network (VPN) service to a separate outside hosting facility 144, which may be located anywhere in the world. The VPN service provides secure communications for the protection of health data over the public Internet. The hospital server system 142 may include a patient management server 150 and a lab server 152. The separate outside hosting facility 144 may include a monitor and control server 146 and database 148 and utilize Internet connections through firewall 154 to firewall 156 to the selectable hospital server system 142, for example. The remote monitoring and control system 140 may also suitably include one or more nursing station terminals 162, a patient bedside terminal 164 associated with each assigned patient 160, a blood testing device for reading blood glucose levels, such as a glucometer 166, and an intravenous IV pump 168. Each of these units may be directly or indirectly connected to the monitor and control server 146 over network connections, such as provided through a switch 158 (or similar device such as a router), through firewall 156 to firewall 154 to the monitor and control server 146. It is noted that depending on the size of an installation, the functions of the patient management server 150 and the lab server 152 may be combined in a single server running separate program threads for each function. It is also possible that the monitor and control server 146 and database 148 may be combined in a single server as well.

FIG. 1C illustrates an exemplary handheld system based monitoring and control system 170 in accordance with an embodiment of the present invention. The handheld system based monitoring and control system 170 may be organized with a hospital server system 174. The hospital server system 174 may include a monitor and control server 176, a database 178, a patient management server 180, and a lab server 182 with the servers 176, 180, and 182 coupled through a switch 184, or similar device such as a router, for communication purposes. The handheld system based monitoring and control system 170 may also suitably include a nursing station terminal 192, a handheld computing device 194 providing the facilities and services of bedside terminal, such as, the bedside terminal 124 and associated with each assigned patient 190, a blood testing device for reading blood glucose levels, such as a glucometer 196, and an intravenous IV pump 198. Each of these units may be directly or indirectly connected to the monitor and control server 176 over network connections, such as provided through a wireless access point 188, and switches 184 and 186, for example. It is noted that depending on the size of an installation, the functions of the monitor and control server 176, database 178, the patient management server 180 and the lab server 182 may be combined in a single server running separate program threads for each function.

While server based monitor and control processes are shown in FIGS. 1A, 1B, and 1C, it is noted that these monitor and control processes could be implemented as one or more programs on a laptop computer or on a handheld computing device with sufficient storage capacity and communication capabilities to access the bedside terminals, nursing stations, patient data, and lab data.

Patient monitoring and blood glucose (BG) control systems, methods, and computer program products in accordance with the present invention as described herein enhance the ability of medical staff to manage patients on an insulin infusion protocol, providing patients with improved blood glucose control. These programs may advantageously operate to accumulate and evaluate data from a plurality of databases and from real time data, either manually or automatically entered, that may be taken at a patient's location. Based on this data, calculations are performed for determining risk situations, patient status, and recommendations in patient care. This information can be presented to a user in easy-to-understand tables, charts, graphs, and utilizing other suitable techniques, such as providing both audible, visual and tactile warning alerts.

The electronic infrastructure, such as shown in FIGS. 1A-1C, may include location information of servers and patients, for example, and access privileges to databases, servers, and the Internet. For example, the Internet may be used for remote access of specialized information or contact of remote specialists. The operating settings of the devices shown in FIGS. 1A-1C are utilized to tailor the equipment and software programs for the patient situation being monitored and for blood glucose level control as described in further detail below.

A risk assessment process quantities for the medical team possible situations and possible protocols to follow to minimize the risk of a patient's health deteriorating, such as identifying that a patient may be entering into hypoglycemia. By understanding the patient dynamics and insight provided by the risk assessment, the process of establishing a dynamic protocol adapted to the patient and medical team becomes reliable, consistent, and trusted.

According to further aspects of the present invention, methods and systems are described for improving the management of medical procedures for patients assigned to medical protocols, such as glucose control protocols. Some embodiments of the present invention incorporate protocols which typically require data to be input into the system, such as a patient's blood glucose levels, the time these levels were taken, whether or not the patient has diabetes and if so, whether the diabetes is insulin-dependent or non-insulin dependent, any medications the patient is taking, and the like. Outputs of the present invention may include instructions for administering insulin and other drugs to the patient or requests for additional data. In some embodiments, outputs may also include alerts to staff to administer drugs, take blood glucose readings, or otherwise provide additional data to the system.

In one embodiment, a patient monitor and control system, such as the systems 100, 140, and 170, receives input in the form of glucose levels and patient related data. Outputs include recommendations to care providers, requests for additional information, and direct commands to control other medical devices. The patient monitor and control system comprises a computing device, such as the server 106, that is operable to communicate, via a communications network or direct connection, with one or more medical sensors and user terminals, such as the handheld computing device 194. The computing device may communicate with peripheral devices directly or indirectly, via a wired or wireless medium such as the Internet, LAN, WAN or Ethernet, token ring, or via any appropriate communications means or combination of communications means. The patient monitor and control system may include one or more databases, such as database 112, to store the patient medical data, as well as all data related to inputs to and outputs from the system. Such databases may reside on the same machine as the patient monitor and control system, or on remote computers connected via an electronic communication protocol.

The decisions dictated by prior art blood glucose protocols are based predominantly on the patient's blood glucose levels. The systems and methods of the present invention allow blood glucose (BG) levels to be entered in several different ways. BG levels may be manually typed into a text box on a displayed form, for example, at the nurses station 120, bedside terminal 124, or handheld computing device 194. The BG levels may also be automatically transmitted to the patient monitor and control system via a blood testing device, such as the glucometer 126, for example, by a wireless connection for transmission of the BG level of a manually taken blood sample. It can also be expected that blood testing devices may be used that may automatically directly read a patient's blood glucose level upon receiving an electronic command. The command can be initiated, for example, from the server 106, nurses station 120, bedside terminal 124, handheld computing device 194 and the like. Such automatic blood testing devices may also sample the patient's blood glucose level at regular intervals without any external commands. Blood glucose levels may also be retrieved from laboratory systems that are local or remote to the hospital server system 104 based on blood samples taken by a lab technician.

The safety of published prior art blood glucose manual protocols can be improved by analyzing the reported blood glucose levels for any irregular values or patterns. Such irregularities can signal either an alert that there has been an error in the glucose readings themselves, or else that the patient is not responding to the protocol as expected. The patient monitor and control system of the present invention can automatically evaluate irregularities in the glucose levels and issue alerts to care providers, technicians, or any other personnel involved in patient care. Alerts can also be issued to other electronic systems, causing them to respond in some pre-specified manner. Alerts may be a visual indication on a display device, a textual message on a display device, a physical vibration, such as utilized on many cell phones, an audible alert or any such alerting method.

A condition that causes an alert may allow the patient monitor and control system to continue dictating recommendations as usual, may cause a change in a decision process used by the patient monitor and control system for determining its recommendations, may cause the patient monitor and control system to stop dictating recommendations, as well as, cause a focused evaluation that may lead to an escalating alert if the condition has not improved.

For example, if the rate of change of the glucose level exceeds a set threshold, the patient monitor and control system can issue an alert to that effect. The rate of change would be calculated by subtracting the value of the previous reading from the most recent reading, and then dividing the result by the time between the two readings. It is assumed that both readings will be converted to equivalent units if necessary.

Other conditions related to the glucose readings that could trigger an alert in some embodiments of the present invention are readings that are themselves above or below defined thresholds, readings that vary by a defined amount from the previous reading, regardless of the time difference between them, readings that don't reach a defined threshold or range of values within a set period of time, and readings that fail to move in a particular direction, or fail to change at all, after a certain period of time, or a certain amount of insulin has been given.

In one embodiment, the above alerts that could be issued by the patient monitor and control system include text messages displayed on a display controlled by the patient management system where the glucose readings are being submitted, or another display, such as in an intensive care unit (ICU), or in a centralized monitoring location. In some embodiments, alerts can also be sent to pagers, printers, cell phones, or regular telephones. Alerts can also be issued by blinking lights, blinking displays, playing audio through a speaker in the ICU or at a monitoring location and may be issued to one or many user devices. Alerts can also be issued by sending an email to a defined email address, or multiple addresses. Multiple types of alerts can be issued at once. Alerts can identify a particular patient as the subject of the alert, or can be issued anonymously for reasons of privacy. An anonymous alert can be broadcast publicly and require an authorized user to log into a user terminal in order to see which patient the alert applies to. For example, a changing light, or a countdown timer on a terminal, can indicate when some action is required without identifying a particular patient.

The decision on what alerts to send can depend on the risk level of the event that triggered the alert. For example, the patient management system can be configured in some embodiments such that a rate of change in the glucose level greater than a but less than or equal to b would cause a warning message to be displayed on screen, but the protocol would continue its recommendations as usual. However, a rate of change greater than b could cause a message to appear on screen, a light to blink, and a page to be sent to the nursing staff. Further, the patient monitor and control system may stop making protocol recommendations until a nurse confirms that the glucose level entered is correct and the situation causing the alert has been addressed.

A number of published prior art blood glucose manual protocols dictate that the glucose checks occur at well-defined intervals. But the rules are extremely complicated and difficult to follow without human errors. For example, one published prior art blood glucose protocol states that blood glucose should be checked hourly until 3 consecutive values within the target range have been recorded. For example, one target range defined by a particular version of the protocol is specified to be 100-139 mg/dL. Once this range has been achieved, the blood glucose may be checked every 2 hours as long as it remains within the target range. Once the blood glucose level has been within the target range for 12-24 hours, checks may be spaced to every 4 hours if there had been no significant change in patient condition or nutritional intake. This particular protocol did not specify what to do if there has been a change in patient condition or nutritional intake. This protocol also specified that the glucose should again be checked every hour if any of the following conditions occurred: the glucose level moves out of the target range, there is a significant change in clinical condition, or the administration of certain drugs, nutritional supplements or therapies has changed. The glucose check interval should also be reduced to 15 or 30 minutes when the blood glucose drops below certain defined levels and the insulin drip is stopped. In an intense environment, such as an ICU, manually following such complicated directions is prone to many errors due to a lack of flexibility in the timing of blood glucose checks or in the administering of insulin, juice, or dextrose solutions.

In one embodiment, the patient monitor and control system can improve on the published prior art protocols by adjusting the glucose check intervals with greater granularity. For example, during periods of instability in the glucose level, when the rate of an insulin infusion is being increased, the patient monitor and control system can check the glucose level more frequently than described in the published protocol, even if the glucose level is not particularly low. By checking the glucose frequently, it can more quickly determine if the insulin infusion is having the desired effect of bringing the glucose rapidly to the target range. Through rapid feedback, the patient monitor and control system can adjust the insulin rate with more granularity as well.

If the blood glucose drops to low levels and the insulin drip is stopped, one published prior art blood glucose protocol dictates that the glucose check intervals be spaced every 15 minutes until the glucose level rises above a defined threshold, at which point the glucose check interval can be spaced every 1 hour again. Depending on how rapidly the glucose level is rising, in some embodiments, the patient monitor and control system of the present invention can increase the check interval at a slower rate than defined in the published protocol, thereby improving the tightness of the control.

The published glucose control protocols do not generally address subtle timing issues that are considered by the present monitor and control system. A published protocol will dictate various events that should occur at specific times, such as, when blood glucose checks should occur, when an IV drip rate should be changed, and when an IV should be started or stopped. The monitor and control system advantageously adjusts these timings in a safe and practical manner to provide flexibility to the medical staff when emergencies or other events prevent meeting recommended timings.

For example, if a blood glucose check for a patient is scheduled to occur close in time to a change in IV drip rate for the same patient, the monitor and control system determines the timing relationship, automatically adjusts the timings and notifies the medical staff to submit the blood glucose reading and change the IV drip rate at the same time. The alternative, asking the nurse to return to the same area of the ICU within just a few minutes, could be burdensome and frustrating for the nurse. The nurse may have already started on another task, and may not be able to return to the first patient for some time. There is a risk that the IV drip rate change could be delayed, and it would have been better to make the change just a few minutes early while the nurse was at the patient's bedside checking the blood glucose level.

In another embodiment, the monitor and control system recognizes when two or more events for different patients in close proximity to each other are scheduled to occur, and adjusts the alert times slightly so that all the events can be staged in close timing to each other or to occur at approximately the same time, when a care provider is in that area of the ICU. The monitor and control system provides such scheduling based on knowledge of the physical location of the patients in the ICU.

Some published protocols have rules which dictate that an IV drip should be restarted after a defined period of time only if the patient's blood glucose level is above a certain threshold. The way this instruction is described, it appears that the blood glucose level should be checked at the time the IV drip is supposed to be restarted. However, it is possible that the blood glucose level was checked a short time before the IV drip was scheduled to be restarted. The monitor and control system provides flexibility to such a rule to avoid burdening the medical staff in such situations, and also possibly eliminating medically unnecessary glucose checks. For example, if the blood glucose level was checked shortly before the time where the IV drip should be restarted, and it was above the threshold at that time, the system may allow the IV drip to be restarted without requiring another blood glucose check. Alternatively, if the blood glucose level was below the threshold shortly before the IV was to be restarted, the system may have a rule to not recheck the blood glucose again at the original IV restart time, but instead wait a longer period of time, such as an hour, before rechecking the blood glucose level, and only then restart the IV drip if the blood glucose level has risen above the threshold.

The monitor and control system is an advantageous event-driven system as described herein that is designed around the production, detection, consumption of, and reaction to events. Events may start as real-world events which are then converted to electronic messages that are sent to an event processing engine, which is a process implemented as a program running on a computer. Events in such a system are expected to occur at unpredictable times, and so such a system is often ideal for real-world environments such as an ICU where unpredictability is expected.

The process of monitoring a patient's physiological state and generating instructions based on a medical protocol, such as a glucose control protocol, may be implemented as an event-driven system having events such as “glucose reading”, “start insulin drip”, “change insulin drip”, “stop insulin drip”, “administer D50 bolus”, “data request”, “data request filled”, and “clock event”. A “glucose reading” event could be generated, for example, when a nurse enters a patient's blood glucose level into a user terminal. The event would include the glucose level that was submitted. A “start insulin drip” or “change insulin drip” event would be an instruction from the system to a medical staff member to change the insulin drip rate and would include the drip rate. A “data request” event could be generated by the system when it needs to ask a question, such as whether or not a patient is diabetic or whether or not a drip was started. A “data request filled” event could be generated when the nurse answers the question thereby fulfilling the data request. In fact, it is important for the system to confirm when a drip was changed instead of assuming the change actually occurred.

A “clock event” could be generated regularly by the system itself in order to mark the passage of time and thereby move the state of the system forward in the absence of other events. For example, the monitor and control system includes a “clock event process” that runs independently and generates clock events. The clock event process may be implemented on the monitor and control server, on another server, or even in the user terminals themselves. The user terminals also have a process running that automatically sends a clock event to the server at regular specified intervals, such as every second, or every minute, and the server sends back updated information to the terminal. Such a process—a “user terminal request”—would keep the terminals updated with current information. This process may also be named an “auto-refresh” process, since the user terminal is automatically causing its displayed information to be refreshed regularly. Other events may include requests to monitoring equipment programmed to respond to these events without user intervention. For example, an “increase drip rate” event could be sent directly to an IV pump, causing it to change the drip rate without nursing intervention.

The user terminal may be a web browser, such as Internet Explorer® or Firefox®, or possibly a Windows Form application, or some other application that provides a user interface that allows a user to select objects and actions, such as selecting a link to a new page of information, such as a patient information detail page, or selecting a file for processing. If the browser is displaying information about a single patient, a user may request information specific to that patient, such as a history of the glucose readings and insulin rates for the patient, or when the next glucose reading is due for that patient. If the browser is on a summary page of some or all the patients being monitored in the ICU, for example a user may request summary information about some or all the patients in the unit. Such information may include, for example, who is in the unit, what specific glucose control protocols apply, whose blood glucose reading is due next, and the like.

The user terminal request may be generated by a JavaScript® program, for example, in a hyper text markup language (HTML) page being displayed on the browser. A monitor and control server, such as one of the servers 106, 146, 176, is configured to generate a JavaScript® program with a particular time period that is sent to the user terminal over hypertext transport protocol (HTTP). The user terminal requests cause the user terminal, such as a bedside terminal 124 or nursing station terminals 122 of FIG. 1 to request, or “pull”, information from the server. Alternatively, the server could “push” updates to the user terminals automatically without the user terminal initiating the transfer of information.

A monitoring and control system, such as system 100 of FIG. 1A is intended to generate recommendations and communicate these recommendations to care providers in a timely manner by displaying them on a display, such as on a patient bedside terminal 124/164 or nursing station terminals 122/162 of FIGS. 1A and 1B or the handheld computing device 194 of FIG. 1C. The timing of the requests and the ability to refresh display screens are configurable through system properties, such as may be set up on the monitor and control server 106, for example. As an example, the pertinent details of a patient's condition may be displayed on a patient detail screen having a number of selectable action buttons. For example, one button may be used to lock the patient detail screen on the bedside terminal. When a screen is locked an auto-refresh command causes the patient detail screen to be refreshed and updated with the latest data available for that patient. In a similar manner, an action button may be located on the locked screen to unlock the screen which on auto-refresh returns the screen to a summary page. In this way, user terminals at each patient's bedside can be locked to show only information for the nearest patient.

Additionally, any alerts related to that patient, such as an alert to check the patient's blood glucose level, would also emanate from that terminal. This arrangement is desirable particularly for audible alerts, since it is helpful to the medical staff for any audible alerts to come from the general area in the ICU where the alert should be resolved. In the case of a blood glucose check or an IV drip rate change, the nurse needs to be near the patient to accomplish these tasks. It would not make sense for an alert for patient A to emanate from a terminal near patient B if patient B was not near patient A. However, if patient B was near patient A, and the terminal near patient A was not working, the monitor and control system may issue an alert for patient A on patient B's terminal. Alternatively, if patient A's terminal were not working and there was no other terminal near patient A, then the monitor and control system may advantageously issue an alert on any active terminal, even one not in close proximity to patient A. In a system where the user terminals are using an auto-refresh process to generate clock events on the central server, the monitor and control process may keep track of how often each patient's data is being requested by a user terminal. The monitor and control system is able to determine whether any patient on a protocol is not being monitored if it did not receive a request for each patient's own information within a certain period of time, which could be configurable. For example the monitor and control system could be configured that it should expect a request for each monitored patient's information at least once every 5 minutes. If it did not receive a request for a particular patient's data within 5 minutes of the previous request, the monitor and control process would know that that patient was not being monitored and would issue an alert to a user terminal nearby to the patient to request that the operation of the suspect monitoring station be checked. If it still determined that the patient was not being monitored a short time later, it could issue an alert to other terminals, or else send a message by pager, phone, or the like, or else alert the medical staff by other means such as by blinking a light.

In one embodiment, a first process is operative on the monitor and control server, such as server 106 of FIG. 1, to process responses from the user terminals and a second process, also operative on the monitor and control server, ensures all active patients on protocols are being monitored by a user terminal, such as the bedside terminal 124 for the safety of the patients. For example, if a patient is started on a protocol and then the bedside terminal that is monitoring the patient and issuing glucose reading alerts goes down, the medical staff would not know when glucose readings were due and the patient could easily be off the protocol. The second process is operative to check each patient monitoring station by polling the terminals and verifying responses are received by the first process. If the second process determines that one of the patient monitoring stations is not operating correctly, escalating alerts may be sent, for example, to the nursing station terminals 122 and may also be sent to other patient terminals, pagers, email stations outside of the ICU, or even trigger a phone call with a recorded alert message.

With this event-driven system, multiple user terminal requests may occur at the same time, and operate separately and in parallel in the system. Each patient is monitored and controlled by a serial stream of events, while the states of different patients can move forward at the same time or at different times as appropriate for each patient. Advantageously, each patient in an ICU, or other such environment with multiple patients, may be safely monitored and controlled while being under the patients own unique protocol and be in a different state within the protocol as compared to the other patients.

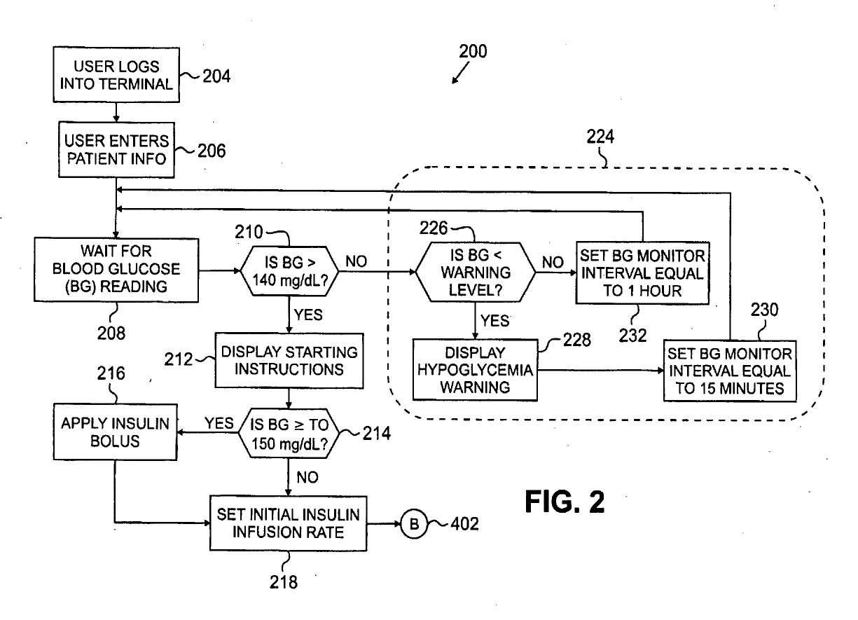

FIG. 2 illustrates an exemplary prestart process 200 in accordance with the present invention. One protocol, the Yale Infusion Protocol, which is one of many such blood glucose control protocols, is adapted for use to demonstrate the advantageous aspects of the present invention. The Yale Infusion Protocol is a manual protocol intended for use in an ICU setting. The Yale Infusion Protocol supports two target ranges, one with a target range of 100-139 mg/dL BG level and another tighter control protocol for a target range of 90-120 mg/dL. The 100-139 mg/dL protocol was published as “Implementation of a Safe and Effective Insulin Infusion Protocol in a Medical Intensive Care Unit” by P. A. Goldberg, et al., Diabetes Care, Vol. 27. No. 2, February 2004 and is incorporated by reference herein in its entirety. The 90-120 mg/dL protocol was published as “Clinical Results of an Updated Insulin Infusion Protocol in Critically Ill Patients” by P. A. Goldberg, et al., Diabetes Spectrum, Vol. 18, No. 3, 2005 and is incorporated by reference herein in its entirety. The monitor and control program uses one process that is adaptable to either target range protocol through the use of BG range tables as described in more detail below. A user may be, for example, a blood lab technician, a nurse, a doctor, or a combination of these individuals. The monitor and control system 170 of FIG. 1C is used in describing the prestart process 200 with a monitor and control program operating from the monitor and control server 176.

The Yale Infusion Protocol recommends a blood glucose level below which a patient should not be started on the protocol, the “starting threshold”, and as a matter of medical judgment each hospital may define its own threshold for starting the protocol. The exemplary system described allows a patient's blood glucose levels to be submitted to the system as soon as the patient is admitted to the ICU. If the submitted values are below the starting threshold, the system remains in a “prestart” state until the blood glucose levels are above the starting threshold.

The prestart process 200 begins at step 204. At step 204, a user logs into a terminal, such as the handheld computing device 194 of FIG. 1C. At step 206, the user then enters or causes to be entered a patient's pertinent information including name, location, the particular insulin infusion protocol to be used, and the like into the monitor and control program. At step 208, a blood glucose (BG) reading is performed, such as by using the glucometer 126, and the results of the reading are submitted by wireless access to the monitor and control server 176 and the lab system server 182. At step 210, the monitor and control program determines whether the BG is greater than 140 mg/dL. If the BG is less than 140 mg/dL, then the process proceeds to step 212. At step 212, starting instructions for the protocol are displayed. For example, the instructions for initiating an insulin infusion may be displayed on the handheld computing device 194. Such instructions may include a first step, such as: INSULIN INFUSION: Mix 1 unit regular human insulin per 2 cc 0.9%. Administer via infusion pump at rates indicated. A second step may also be displayed, such as: PRIMING: Flush 20 mL of infusion through all IV tubing before infusion begins, to saturate the insulin binding sites in the tubing. At step 214, a determination is made whether the BG is greater than or equal to 150 mg/dL. If the BG is greater than or equal to 150 mg/dL then an insulin bolus is recommended. A bolus is medication or compound that is given to a patient to raise the concentration of a particular blood component, such as the level of blood glucose. At step 216, an insulin bolus is prepared to be applied to the patient. For example, the insulin bolus may be given intravenously by a nurse using a syringe. At step 218, the initial drip rates are displayed by the monitor and control program on the handheld computing device 194 and set up on the IV pump 198. At this point, the pre-start process proceeds to point B 402 in the main protocol process 400 to be described in detail below. Returning to step 214, if the BG was determined to be less than 150 mg/dL, the prestart process 200 proceeds to step 218 with a different insulin infusion rate setting displayed and set up on the IV pump 198.

Returning to step 210, if the BG was less than or equal to 140 mg/dL, the prestart process proceeds to warning and interval control process 224. Each published protocol defines a blood glucose threshold as a warning level below which a Dextrose 50% (D50) solution may be given in order to reverse hypoglycemia. D50 is a 50% solution of dextrose in water that may be given as an intravenous bolus to patients who have hypoglycemia. For the 90-120 protocol, the warning level is 70 mg/dL and for the 100-1390 mg/dL protocol the warning level is 75 mg/dL. During the prestart period, the Yale Infusion Protocol does not make recommendations for therapy prior to reaching the starting threshold level. Rather than ignore patients experiencing hypoglycemia during the prestart period, the warning and interval control process 224 advantageously determines if a hypoglycemia warning should be issued and also determines an appropriate check BG interval. In this case, the monitor and control process is not dictating instructions, but merely alerting the medical staff that they should use their medical judgment with regard to the hypoglycemic blood glucose level. This alert is made to shorten the blood glucose check interval as a safety measure so that the medical staff will not inadvertently ignore the patient for a long period. For example, at step 226, it is determined whether the BG is less than a warning level, such as, the 70 mg/dL or 75 mg/dL levels. If the BG is less than the warning level, the prestart process 200 proceeds to step 228. At step 228, a hypoglycemia warning is displayed to alert the user that the BG level is low and that a doctor should be notified. The prestart process 200 remains at step 228 until a user submits the responsible doctor's name, to ensure that a medical staff member has been informed of the hypoglycemia. At step 230, BG readings are scheduled every 15 minutes until the readings are above the hypoglycemic threshold. The time interval between readings is configurable and sufficiently short to ensure that the medical staff is checking the patient's blood glucose levels appropriately following a hypoglycemic reading. Returning to step 226, if the BG level is determined to be equal to or above the warning level threshold, the prestart process proceeds to step 232. At step 232, BG readings are scheduled every hour until the readings are above the 140 mg/dL level as checked at step 210.

The monitor and control program alerts the nursing staff to submit BG readings at defined intervals as specified in a particular protocol. However, given the uncertainty of an ICU, the monitor and control program provides flexibility for the submission of BG readings at other times, such as before and after the protocol's scheduled time. After a BG reading has been taken it should be entered as soon as possible from when it was taken, preferably within five minutes. Alternatively, the time since the blood glucose was taken can be submitted to the monitor and control program along with the blood glucose reading, so that the monitor and control program may accurately establish the time the blood was drawn from the patient. In general, allowing flexibility in the timing of BG readings is not a problem, since it has been determined that many protocols rely on rate of change calculations that automatically account for the time between BG readings. In addition, where the protocols specify that the insulin infusion should be stopped for a period of time, additional logic may be utilized to account for the possibility of irregular BG reading times, as described in further detail below.

Although protocols generally dictate insulin infusion rates, the monitor and control program advantageously allows the medical staff to adjust the infusion rates. Whenever a change of infusion rate is recommended, the monitor and control program requests the medical staff to enter the actual infusion rate that was set. The monitor and control program relies on the submitted infusion rates for any calculations instead of assuming the recommended rates were actually used. For any deviation from the recommended rate, the user is asked to submit a reason, which is shown on the user terminal's on-screen record and any printed activity reports.

In one embodiment, the blood glucose measurement may be taken by a nurse, doctor or technician and entered into a data input device, such as a keyboard, of the patient monitor and control system. In another embodiment, the blood glucose measurement may be communicated to the patient monitor and control system automatically from a sensor attached to the patient. Any response to the blood glucose levels, such as adjusting an insulin drip, may be accomplished by the same person who took the BG reading, a different person at a later time, or possibly through direct control of an insulin IV pump or other drug delivery device. Such adjustments may also depend upon the individual's authorization level. For example, a blood technician may not have the training and authorization to change settings on an insulin IV pump. Therefore, in some embodiments of the present invention, it is important to maintain a separation within the system between the blood glucose measurement, which may belong to a first set of medical procedures with a first level of authorization, and the response to the measurement, which may include medical procedures belonging to a second set of medical procedures which may have a different level of authorization than the first level of authorization. In some embodiments, such a separation of concerns might also be necessary for other types of processes that could involve multiple people with different roles and responsibilities. To ensure a recommended insulin rate change request actually occurred, the system issues a request for confirmation that the insulin rate was changed. Further, the confirmation may only be submitted by someone with sufficient authorization which may not include a technician who did the blood test. If a confirmation is not received within a specified period, an escalating alert process is initiated until confirmation is received. The confirmation includes the time of the rate change and whether the recommended rate change was followed or was modified. If the recommended rate change was not followed, for example, a reason must be entered with the confirmation.

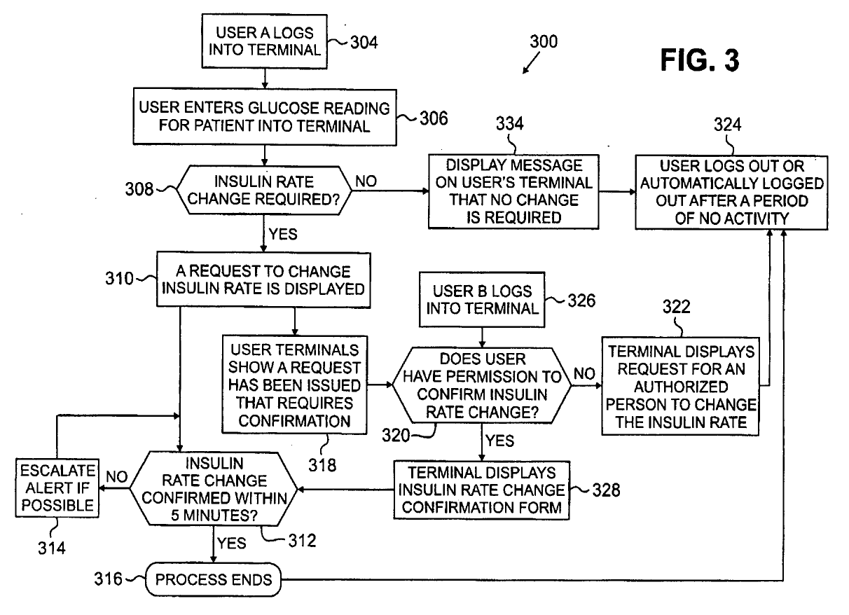

For example, FIG. 3 illustrates an exemplary separation of concerns decision in an insulin rate adjustment process 300 in accordance with the present invention. In FIG. 3, a first user, user A, may be a technician who draws blood and records blood glucose levels with a handheld glucometer but does not have permission to change the insulin infusion rate. At step 304, the user logs into a terminal such as the handheld computing device 194 of FIG. 1C. At step 306, the user takes a blood glucose reading and enters the reading into the handheld computing device 194 which transmits the BG reading to the monitor and control program running on server 176. The monitor and control program determines at step 308 whether an insulin infusion rate change is required. If an insulin rate change is required, the process 300 proceeds to step 310. At step 310, a request to change the insulin infusion rate is displayed on the handheld computing device 194, for example, and at the nursing station terminal 192. The process 300 proceeds to both steps 312 and 318. At step 312, a determination is made whether the insulin infusion rate has been changed and confirmed. If the insulin infusion rate has not been changed and confirmed, the process 300 proceeds to step 314. At step 314, an alert is given and escalated if possible. For example, as a first alert, the rate change message is displayed as a flashing message on the handheld computing device 194. If no confirmation is received after a pre-specified period, such as 2 additional minutes, the handheld computing device 194 provides an audible alarm along with the flashing rate change message. If another programmed time interval has passed without confirmation, the audible alarm is increased in volume. If another programmed time interval has passed without receiving a confirmation, a message is sent to the nursing station terminal 192. The audible alerts continue gaining strength until the alert is responded to. For example, if after another programmed time interval the confirmation has still not been received, all terminals for all patients give an audible warning sound which continues to escalate in volume until confirmation resolution is achieved. Once confirmation has been achieved, at step 312, the process proceeds to step 316 and ends the rate change process and proceeds to step 324 where the user may log out.

Returning to step 318, a rate change request message is displayed on some or all of the user terminals. For example, the rate change request may be displayed on the terminal where the BG reading was entered, and additionally on all terminals that are displaying a summary of all patients in the ICU, but not necessarily on user terminals that are locked to a different patient. Alternatively, all terminals may display a rate change request, but such a request may be less prominent on a user terminal locked to a different patient so as not to imply that the request was for that particular patient. Alternatively, only user terminals in the vicinity of the patient to whom the request is directed would display the rate change request. At step 320, if only user A, as a blood technician with no authority to make insulin infusion rate changes, is logged into a user terminal that is active to the monitor and control program, the process 300 proceeds to step 322. At step 322, one or more terminals display a request message for an authorized person to change the insulin rate. At step 324, user A logs out or is automatically logged out due to a period of no activity.

Due to the displayed message, a different user, user B, such as a doctor who has authorization to make insulin infusion rate changes, logs into a terminal at step 326. At step 320, it is determined that user B has authority to make insulin infusion rate changes and the process 300 proceeds to step 328. At step 328, a terminal used by user B, such as another handheld computing device, displays an insulin rate change confirmation form. Thus, a doctor can advantageously review the necessary data and confirm the change either locally, for example, in an intensive care unit responsible for the patient, or remotely based on data accessible to a computing device. If the rate change is not confirmed, the process 300 proceeds to step 312 and follows the process noted above to alert the user and nursing staff if an insulin rate change has not been confirmed within 5 minutes.

Returning to step 308, if an insulin rate change is not required, the process 300 proceeds to step 334. At step 334, a message is displayed on the handheld computing device 194 that no change is required. At step 324, either the user logs out of the program running on his handheld computing device 194 or the user is automatically logged out after a period of no activity.

The separation of concerns may be based on a user authentication process. For example, the system may have two or more levels of user authentication, such as at least a primary level and a temporary level. A primary user may be required to initially login to the system. The temporary user can then authenticate and temporarily override the primary user authentication, as described in more detail below. In order to allow flexibility with multiple medical staff, logins that occur while a temporary user is active replace the current temporary user with a new temporary user. When a presently temporary user logs out, the primary user regains control. When the primary user logs out, only the initial login screen is displayed. This level of control allows for a user terminal to remain in a low-permission state most of the time so that ICU staff can view patient information and action alerts. Activity on the system is set up to require a user with additional permissions to temporarily login to a user terminal. The temporary login has a short timeout delay for safety; in other words, if the temporarily logged in user does not perform any activity on the terminal within a relatively short period of time, such as 2 minutes, that user is automatically logged out of the terminal and the primary low-permission login regains control. In order to provide this level of control, the primary and temporary users utilize a username and password in order to login. The temporary user has an independently configurable timeout delay, such as 2 minutes of inactivity. The primary user typically remains logged in until manually logged out. Whenever the currently active user changes, if the new user has permission to view the current screen, then the same screen is displayed for the new user. Whenever the currently active user changes, if the new user does not have permission to view the current screen, then a default screen that the user does have permission to view is displayed. If the screen has a request pending for patient data and the new user does not have permission to submit the requested patient data, then the data request remains visible but disabled, and a message is displayed to notify a user who does have permission to respond to the data request, such as a nurse or doctor. If the screen was showing a disabled data request and a new user with permission to submit the patient data logs in, then the new screen shows the enabled data request.

In some embodiments, a hospital may not even want the system to remain in a low-permission view-only state. In those cases, the primary user timeout can be set to a short delay so that the system generally remains on a login screen. In order to use the system at all, a user with sufficient permissions must log into the login screen. Once done using the system, or after a short period of inactivity, the user would be logged out of the system and the system would again return to just showing the login screen.

In such an embodiment, the system can present a monitoring display to the medical staff that has no identifiable patient information on it, and that is viewable even when no user is logged into the terminal. Such a display can indicate the time remaining until some unidentified patient requires an intervention, such as a blood glucose reading; or that some unidentified request is currently pending. A user seeing such an alert would know to log into a terminal in order to view the specifics of the alert, such as which patient the alert is for.

As an alternative to a display, the system may simply issue audible alerts that indicate when some action is required by a user. For instance, a particular sound may indicate that an action is required within 2 minutes, and a different sound can indicate that an action is required immediately. Such alerts are anonymous enough to not require user authentication in order to view or hear them. Such alerts may also emanate from audio devices, such as the alert device 125, located in or near the patient's room, and not from a user terminal.

The system can also issue alerts to an alert device, such as the alert device 125, using colored lights located near a patient's room, such as above a patient's door. For example, a yellow light might indicate that an action for the patient in the room is required within 2 minutes, and a red light can indicate that an action is required immediately. Such alerts would presumably not require user authentication.

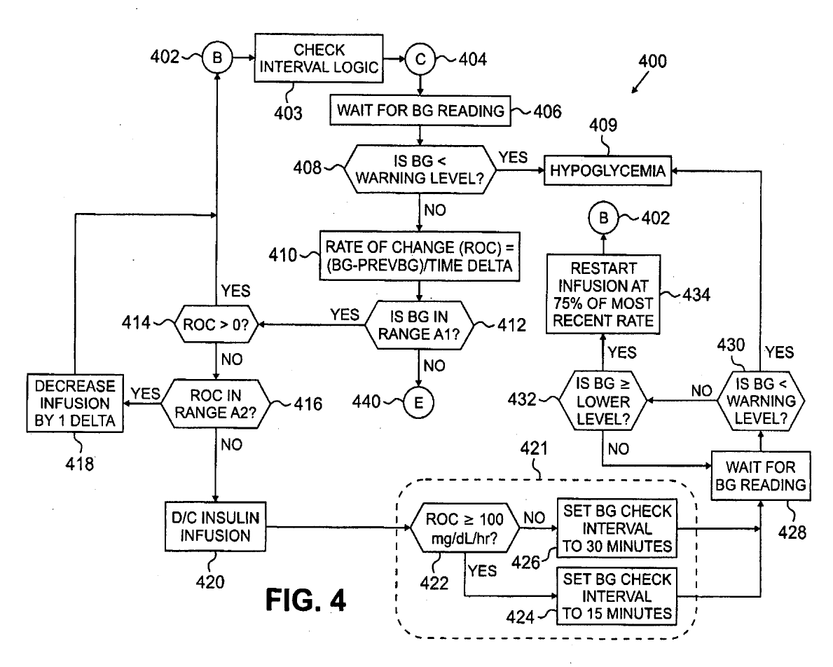

FIG. 4 illustrates an exemplary main protocol process 400 in accordance with the present invention. The main protocol process 400 is responsible for BG levels equal to or above the hypoglycemic warning level, such as the warning levels of 75 mg/dL for the 100-139 mg/dL protocol or 70 mg/dL for the 90-120 mg/dL protocol. The two versions of the protocol use a table, such as Table 1 below, governing actions to be taken depending on the BG levels.

TABLE 1

| Column 1 | Column 2 | Column 3 | Column 4 | |
|---|---|---|---|---|
| RANGE A1 | RANGE B1 | RANGE C1 | ELSE | INSTRUCTIONS |
| | | RANGE C2 | ROC > 0 | ↑ INFUSION BY “2Δ” |
| | RANGE B2 | RANGE C3 | RANGE D1 | ↑ INFUSION BY “Δ” |
| ROC > 0 | RANGE B3 | RANGE C4 | RANGE D2 | NO INFUSION CHANGE |
| RANGE A2 | RANGE B4 | RANGE C5 | RANGE D3 | ↓ INFUSION BY “Δ” |
| STEPS 8-15 | ELSE | ELSE | ELSE | HOLD 30 LOGIC |

Table 1 uses the columns of the table to define various ranges of BG levels that are associated with each protocol and the rows specify recommended actions in the instructions column for a particular grouping of BG ranges which are rate of change ranges. In the instructions column, the “Δ” symbol specifies a rate of change in units/hour and the “2Δ” symbol is equal to twice the “Δ” rate of change. For example, in the fourth row having “Range A2” in column 1, the insulin infusion should be reduced “↓” by “Δ”. Another table specified in the protocol documentation (not shown) gives the actual BG values in the various ranges. For example, “Range B4” is given in mg/dL/hour and for the 90-120 protocol, “Range B4” is specified as −40≦x≦−20 mg/dL/hour. Another table specified in the protocol documentation (not shown) gives for various current insulin infusion rates what values of “Δ” and “2Δ” should be applied according to the instructions in Table 1. For example, if the current insulin infusion rate is 3-6 units/hour the value of “Δ” is 1 unit/hour indicating the current rate should either be increased “↑” by 1 unit/hour or decreased “↓” by 1 unit/hour according to the instructions in Table 1.

In the main protocol process 400 of FIG. 4, BG monitor times are the recommended BG reading times. Due to the busy environment of an ICU, BG readings may be taken at times other than the recommended times and the process 400 advantageously allows for this by waiting until an actual reading has been entered. For example, a recommendation is made to “check the BG in 30 minutes” according to the published protocol however, the reading may be actually taken either before the 30 minutes, such as at 26 minutes after the notification or after the 30 minutes such as at 37 minutes. Delayed BG readings may be noticed by a separate process that issues alerts to the medical staff to check the BG.

In the monitor and control process, treatment recommendations are given, based on one of the published protocols. For example, a treatment may be recommended to administer 1 AMP (25 g) of D50. The monitor and control process advantageously allows for variations in the treatment recommendations and in this example, the monitor and control process allows a user to choose to (1) confirm 1 AMP of D50 has been given, (2) to confirm no D50 was given and provide a reason, or (3) to confirm a different amount of D50 was given and provide a reason.

At any time a recommendation is given, the process 400 generally enters a wait state waiting for the user to respond. During these wait states, an authorized user can choose to either stop the protocol or override the insulin infusion rate recommendation. Also, an independent process will inform users whether a BG reading was not provided at the recommended interval, or whether some other request from the system has not been addressed.

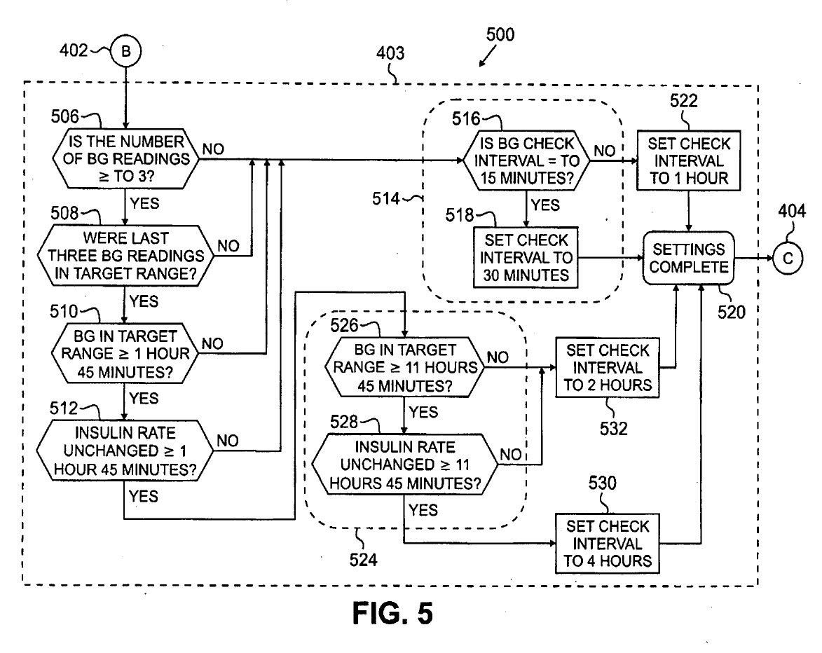

The process 400 begins at point B 402 with entry into a check interval logic step 403 which initializes a countdown clock for each patient providing timing support for BG reading alerts to the medical staff. Step 403 determines the time from when the last BG reading was submitted until the next BG reading is due. The time interval between BG readings is referred to as the blood glucose check interval. The check interval logic 403 is responsible for supporting protocol instructions that are based on the stability of BG readings. For example, protocol instructions may be given to check BG hourly until stable within a target range for three consecutive readings. Once a BG reading is considered stable, instructions may be given to check the BG every two hours. If the BG readings are considered stable for twelve to twenty-four hours, instructions may be given to consider checking every three to four hours if there has been no significant change in clinical condition or nutritional intake. Protocol instructions to resume hourly BG readings may also be given if any of the following situations occur. For example, a patient experiences a change in insulin rate, a significant change in clinical condition, an initiation or cessation of steroid or pressor therapy, an initiation or cessation of dialysis or continuous veno—venous hemofiltration (CVVH), or an initiation, cessation, or having a rate change of nutritional support. The check interval logic 403 is discussed in further detail in connection with FIG. 5 below.

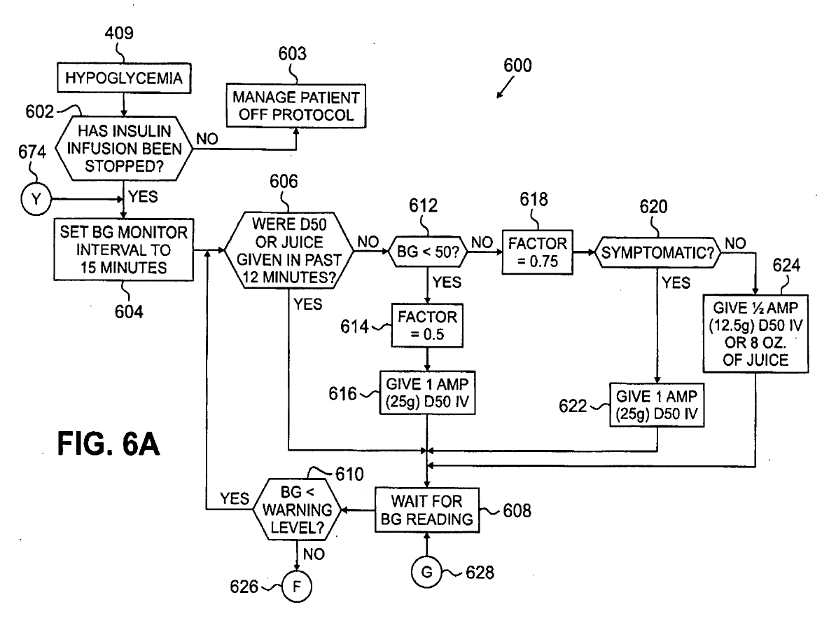

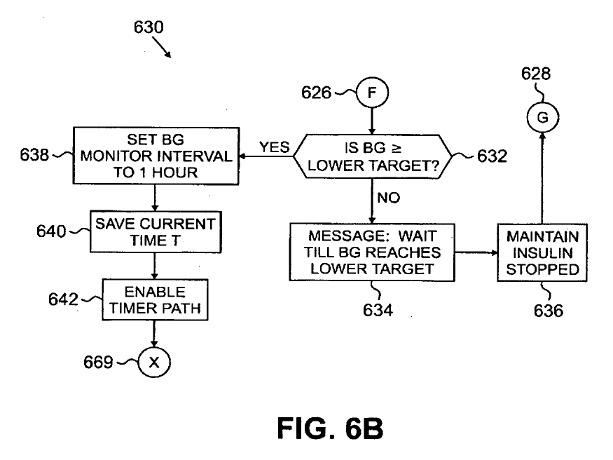

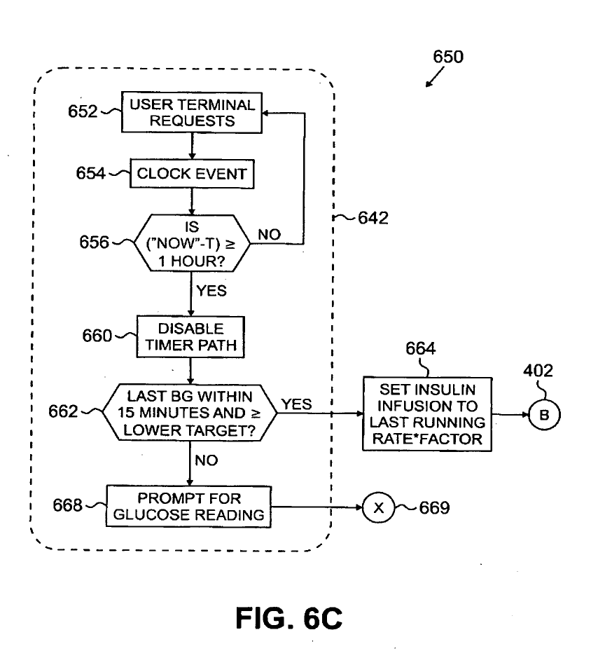

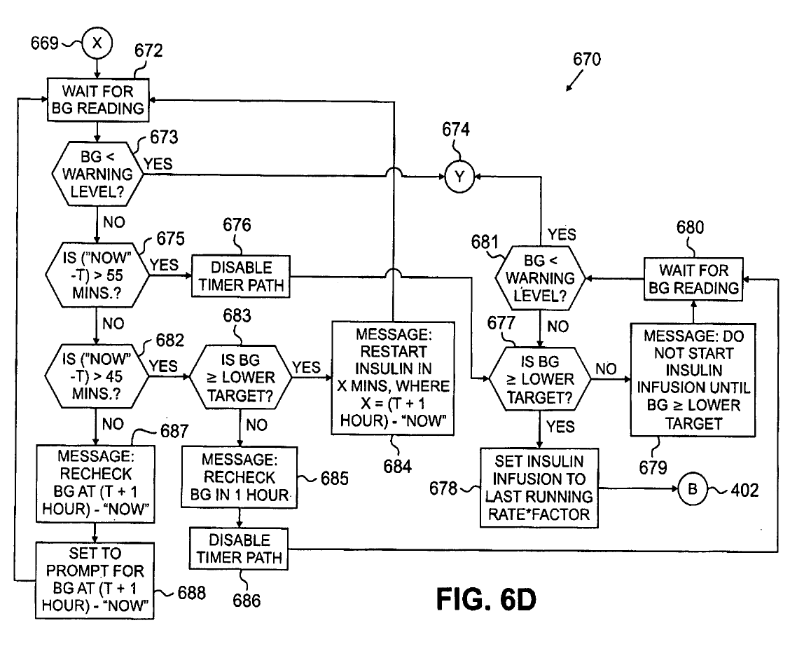

The check interval logic step 403 exits to point C 404 which is the entry point to submit a blood glucose (BG) reading in step 406. At step 408, a determination is made whether the BG is less than the warning level for the operative protocol. If the BG is less than the warning level, then the process 400 proceeds to step 409 to respond to hypoglycemia as described in more detail with regard to FIGS. 6A-6D.

Returning to step 408, if the BG is greater than the warning level, then the process 400 proceeds to step 410. At step 410, the rate of change (ROC) in BG readings is calculated. The ROC is equal to the present BG reading minus a previous BG reading (PrevBG) divided by the time between readings (time delta). A warning note is displayed that if the current BG reading was taken more than five minutes ago, the BG level must be rechecked before submitting.

At step 412, a determination is made whether the BG is in Range A1, which, for example, in the 90-120 mg/dL protocol is 70≦x≦89 mg/dL and for the 100-139 mg/dL protocol is 75≦x≦99 mg/dL. If the BG reading is in Range A1, the process 400 proceeds to step 414. At step 414, a determination is made whether the ROC calculated in step 410 is greater than zero. If the ROC is greater than zero the main protocol logic proceeds to step 402 the entry point into the check interval logic 403. If the ROC is not greater than zero, then the process 400 proceeds to step 416. At step 416, a determination is made whether the ROC is in range A2, which, for example, in the Yale 90-120 mg/dL protocol is −20≦x≦0 mg/dL/hour and for the Yale 100-139 mg/dL protocol is −25≦x≦0 mg/dL/hour. If the ROC is in the range A2 for the operative protocol, then the process 400 proceeds to step 418. At step 418, a recommendation is given to decrease the insulin infusion rate by 1 delta. The process 400 then returns to step 402 the entry point B to the check interval logic 403.

At step 416, if the ROC is determined to not be in range A2, the process 400 proceeds to step 420. At step 420, the insulin infusion is confirmed to be stopped. The process 400 then advantageously proceeds to a change check interval process 421. In cases where the BG has changed particularly rapidly, a safety feature was added to the protocol of first requiring a 15 minute check, then changing to a 30 minute check, and only then changing back to hourly checks. Although this is a change from the published protocol, the more frequent checks provides added safety to the patient. Thus, at step 422, a determination is made whether the ROC is greater than or equal to 100 mg/dL, hour. If the ROC is greater than or equal to 100 mg/dL/hour, then the process 400 proceeds to step 424. At step 424, the BG check interval is set to 15 minutes and the process 400 proceeds to step 428. At step 422, if the ROC is less than 100 mg/dL/hour, then the process 400 proceeds to step 426. At step 426, the BG check interval is set to 30 minutes and the process 400 proceeds to step 428.

At step 428, the BG levels are read and submitted and at step 430 a determination is made whether the BG is less than the warning level and if less than the warning level, the process 400 proceeds to the step 409 for hypoglycemia. At step 430, if the BG reading is not less than the warning level, then the process 400 proceeds to step 432. At step 432, a determination is made whether the BG is greater than or equal to a lower level, which, for example, in the Yale 90-120 mg/dL protocol is ≧100 mg/dL and for the Yale 100-139 mg/dL protocol is ≧90 mg/dL. If the BG is not ≧lower level, then the process 400 returns to step 428 to recheck and submit the BG levels. If the BG is ≧ the lower level, then the process 400 proceeds to step 434. At step 434, the insulin infusion is restarted at 75% of the most recent rate. After step 434, the process 400 proceeds to step B 402 which is the entry point to the check interval logic 403.

Returning to step 412, if the BG reading is not in range A1, then the process 400 proceeds to point E 440 which is the entry into the logic for BG readings that fall within columns 2-4 of Table 1.

FIG. 5 illustrates an exemplary check interval process 500 in accordance with the present invention. Steps 506 and 508 determine whether three consecutive BG readings are in the target range which is required by the protocol to consider the patient to have stable BG levels. If it was determined in step 506 that there were less than three readings or if one or more of the last three readings was not in the target range, the process 500 proceeds to step 514.

Steps 510 and 512 provide additional criteria to the definition of stable BG levels beyond the published protocols. Since the main protocol process 400 allows BG readings to be submitted at any time, a duration of stability is determined by step 510 to be at least 1 hour and 45 minutes. Three readings submitted exactly 1 hour apart as recommended by the published protocols defines a time interval of 2 hours. By using an interval of at least 1 hour and 45 minutes, the check interval process 500 accommodates a level of uncertainty in BG submission intervals while still being reasonably close to 2 hours. The important point is not that the interval is exactly 1 hour and 45 minutes, but that it is somewhat less than the 2 hours specified in the published protocol, in order to accommodate some flexibility in the actual BG reading times.

Step 512 adds a new requirement to determine whether the insulin infusion rate is stable as well. Where the published protocol recommends hourly monitoring if the insulin rate changes, the check interval process 500 requires hourly monitoring, as described in further detail below.

If any of the steps 506, 508, 510, and 512 are determined to be negative, the process 500 proceeds to an advantageous set check interval logic 514. At step 516, a determination is made whether the BG check interval is set to fifteen minutes, which may have been set for example at step 424 in FIG. 4. If the BG check interval is set to fifteen minutes the process 500 proceeds to step 518. At step 518, the check interval can be set more coarsely to thirty minutes providing more caution when coming from a fifteen minute check interval situation. At step 520, the check interval settings are complete and the process 500 proceeds to point C 404.

Returning to step 516, if the BG check interval is not equal to fifteen minutes, then the process 500 proceeds to step 522 where the check interval is set to one hour. After setting the check interval to one hour the process 500 proceeds to step 520 and then to point C 404.

If the steps 506, 508, 510, and 512 are all positive, the process 500 proceeds to advantageous stable check interval logic 524. The published protocol recommended time for determining stability of the BG readings is greater than or equal to twelve hours. At step 526, an advantageous determination is made whether the BG is in the target range for greater than or equal to eleven hours and forty five minutes. By using an interval of 11 hours and 45; minutes, the check interval process 500 accommodates a level of uncertainty in BG submission intervals while still being reasonably close to 12 hours. At step 526, if the determination is made that the patient's BG level was stable in the target range for at least eleven hours and forty five minutes then the process 500 proceeds to step 528. At step 528, an advantageous determination is made whether the insulin rate has remained stable for at least eleven hours and forty five minutes for reasons of BG reading time flexibility as described above. At step 528, if the determination is made that the patient's insulin rate has remained stable for at least eleven hours and forty five minutes, then the process 500 proceeds to step 530. At step 530 the check interval is set to four hours and then proceeds to step 520 completing the settings and then to point C 404 in process 400.

If any of the steps in the stable check interval logic 524 is negative, then the process 500 proceeds to step 532 which sets the check interval to two hours. At step 520 the settings are complete and the process 500 proceeds to point C 404.

In the main protocol process 400, step 409 begins a hypoglycemia process. For example, FIG. 6A illustrates an exemplary hypoglycemic blood glucose (BG)<a warning level process 600 in accordance with the present invention. All BG readings entered into the system are first compared to the hypoglycemia warning level defined for each protocol, such as step 408 of FIG. 4. For example, a warning level is specified as 75 mg/dL for the 100-139 mg/dL protocol and as 70 mg/dL for the 90-120 mg/dL protocol. Any reading below this level causes the monitor and control program to follow the process 600.

At step 602, a recommendation to stop the insulin infusion is given. The user has a choice to either confirm that the insulin infusion is being stopped, or else manage the patient off protocol. At step 603, the protocol is stopped and the patient is managed by the medical staff off the protocol. This is an example of allowing the medical staff to use their medical judgment and override the protocol's recommendations. In this case, the protocol must stop since its logic cannot accommodate allowing the insulin drip to remain running when the patient is hypoglycemic. The monitor and control program will not continue making any protocol recommendations unless the user confirms that the insulin is stopped. At step 604, the monitor and control program sets the BG monitor interval to 15 minutes and begins prompting for BG readings every 15 minutes.

At step 606, a determination is made as to whether D50 or juice has been given in the past 12 minutes. Step 606 accounts for the possibility of having BG readings being entered between BG alerts. The published protocols dictate that if the BG is still <50 mg/dl after 15 minutes, another 25 g D50 bolus should be recommended. It has been determined that the monitor and control program should not be too strict about the recommended 15 minute limit. For example, if a nurse submitted a BG<50 mg/dl after 14 minutes, it would be risky not to dictate another insulin bolus, particularly since the next BG reading alert would not occur for another 15 minutes. Consequently, a 25 g D50 bolus is administered on any BG<50 mg/dl as long as it has been at least 12 minutes since any D50 bolus was administered. Twelve minutes is not an absolute requirement, but an example of a time interval that is somewhat shorter than the published value of 15 minutes in order to accommodate flexibility in BG reading times. This situation is another one in which providing flexibility for taking BG readings at times which may vary requires additional process steps not provided for by the protocols, but which are readily provided by the present invention.

At step 612, a determination is made as to whether the BG level is less than 50 mg/dL, and if so a factor of “0.5” is stored at step 614 for later use at the time when the insulin should be restarted, as described at step 664 in FIG. 6C. At step 616, the recommendation to give 25 g D50 IV is made. At step 608, the monitor and control program responds to the next submitted glucose reading. Although the system does not issue an alert until 15 minutes have elapsed since the prior reading, it still responds to any readings entered prior to this alert. At step 610, the BG reading is again compared with the hypoglycemia warning level. If the BG reading is below this warning level, the process repeats from step 606. This loop ensures that continued hypoglycemia will not go untreated.

Returning to step 612, if the BG reading was above the warning level a factor of “0.75” is stored at step 618 for later use at the time when the insulin should be restarted, as described at step 664 in FIG. 6C. At step 620, a determination is made as to whether the patient is symptomatic. If the patient is symptomatic, the process 600 proceeds to step 622 where a recommendation to administer 1 amp of D50 IV is given. If the patient is not symptomatic, the process proceeds to step 624 where a recommendation to administer ½ amp of D50 IV or 8 ounces of juice is given. Both steps 622 and 624 return to step 608 to recheck and submit the BG levels. At step 610, if the BG is above the warning level the process 600 proceeds to point F 626.

FIG. 6B illustrates an exemplary hypoglycemic sub-process 630 in accordance with the present invention. Point F 626 is reached when the BG level indicates the patient is no longer hypoglycemic. At step 632, a determination is made whether the BG is still below the lower target level, such as 100 mg/dL for the 100-139 mg/dL protocol or 90 mg/dL for the 90-120 mg/dL protocol. If the BG is below the lower target, the sub-process 630 proceeds to step 632 and a message is given to “wait till the BG reaches the lower target”. At step 636, the insulin rate is not started and is maintained stopped as previously confirmed at step 602 in FIG. 6A. Further, the process reminds the user to hold off giving insulin. Since the BG monitor interval has not been changed from the 15 minute setting of step 604, the next prompt to take a BG reading causes the sub-process 630 to proceed to point G 628 which returns to step 608 in the process 600 of FIG. 6A to read and submit the BG levels. At step 610, the BG is checked to determine if it is less than the warning level which at this point it should still be and the process 600 proceeds to point F 626 and the sub-process 630 of FIG. 6B. If for some reason, the patient again becomes hypoglycemic, step 610 would detect this condition and the process 600 would return to step 606 to recommend appropriate intervention. If the patient is not hypoglycemic, the loop of steps 632, 634, 636, 608, 610, and back to step 632 is maintained until at step 632 it is determined that the BG is greater than or equal to the lower target which causes the sub-process 630 to proceed to step 638.

At step 638, the BG monitor interval is set to one hour and at step 640 upon entering a one hour waiting period the current time is stored as variable T. At step 642, a timer path is enabled. The process 630 then proceeds to point X 669 which is the entry to hypoglycemic sub-process 670 in FIG. 6D.

FIG. 6C illustrates an exemplary timer path process 650 in accordance with the present invention. The timer path step 642 utilizes user terminal requests as clock events described above, for each patient under a monitor and control protocol to drive the protocol state forward independently for each patient. Clock events may also be generated on the monitor and control server or on another server, for example. At step 654, each user terminal request results in a clock event being submitted to the monitor and control system. At step 656, a determination is made whether the present time minus the stored time T that was stored at step 640 is at least one hour. If the elapsed time is not at least one hour, the process 650 waits for the next browser request and no further processing of the current clock event occurs.

If at step 656, the elapsed time was determined to be greater than or equal to one hour, then the timer path is disabled at step 660 so that further clock events will not enter the timer path step 642 until the timer path is enabled again. At step 662, a determination is made whether the last BG reading was within 15 minutes and if so whether the BG reading was at least at the lower target. This step 662 allows for variability of BG reading times. If a BG reading above the lower target was submitted just prior to the end of the hour waiting period, it is not necessary to require a nurse to take another fingerstick when the hour has passed. This step 662 advantageously allows for a 15 minute window before the hour is up where a BG reading above the lower target will suffice to trigger an insulin restart at the end of the hour interval. At step 664, the insulin infusion rate is set to a new rate that is calculated by multiplying the previous rate by the factor “0.5” stored in Step 614 of FIG. 6A, or a factor of “0.75” stored in Step 618 of FIG. 6A. The process 650 then proceeds to point B 402 which is the entry into the check interval logic 403 of FIG. 4.

If at step 662 it was determined that a BG reading has not been submitted within 15 minutes of an elapsed hour, a prompt is given to take a BG reading at step 668. The process 650 proceeds to point X 669 which is the entry into sub-process 670 of FIG. 6D.

FIG. 6D illustrates an exemplary hypoglycemic sub-process 670 in accordance with the present invention. Point X 669 is an entry point into step 672 where the next BG submission is checked. At step 673, for reasons of patient safety, the BG level is compared to the hypoglycemic warning level. If the BG level indicates hypoglycemia, the sub-process 670 proceeds to point Y 674 which returns to step 604 at FIG. 6A.

The sequence of steps 675-678 is followed in the common case where a BG reading is submitted anytime after 5 minutes before the end of the 1 hour wait period to address the flexibility provided in the timing of BG readings. In this case, the timer path is disabled at step 676, and at step 677 the BG level is compared to the lower target. If the BG is still above the target, then at step 678, the user is prompted to restart the insulin at 50% of the previous rate based on the FACTOR which was set to “0.5” at Step 614 in FIG. 6A, or at 75% of the previous rate based on the FACTOR which was set to “0.75” at Step 618 in FIG. 6A. The process then continues to point B 402 FIG. 4 with the main protocol process 400.

The sequence of steps 677, 679-681 is a loop that is dependent on the BG levels. If at step 677 it is determined that the BG level has fallen below the lower target, then, at step 679, a message is displayed to not start an insulin infusion until BG≧lower target. At this point, it is known that the BG is not in the hypoglycemic range due to step 673. The system then enters a glucose check loop, reading and submitting the BG in step 680, determining if the patient is hypoglycemic in step 681 and returning to step 677. The system is still providing prompts to recheck the BG every hour. Once the BG reading is ≧ the lower target at step 677, the sub-process 670 proceeds to step 678 and restarts the insulin infusion drip and then proceeds to point B 402 FIG. 4 with the main protocol process 400. If at step 681, it is determined that the BG has fallen below the hypoglycemia warning level, then the sub-process 670 proceeds to point Y 674 which returns to step 604 at FIG. 6A.

The sequence of steps 682-684 is followed for the case where a BG reading is submitted between 5 and 15 minutes prior to the end of the 1 hour wait as determined at step 682 to address the flexibility provided in the timing of BG readings. If at step 683 it is determined that the BG is still above the lower target 45 minutes after it first rose above this target level, then there is little concern that the BG will drop below this level within the remaining time prior to the insulin restart. Based on this determination, a message is displayed indicating that the insulin is now set to restart in X minutes, where X is the number of minutes remaining in the hour wait, given by (T+1 hour)−“NOW”, where “NOW” is the present time. Step 662, discussed above, triggers the insulin restart alert at the end of the hour without requiring another BG reading. This is an example where requiring another BG reading may be considered onerous to the medical staff, which may decrease their likelihood of using the system.

The sequence of steps 682, 683, 685, and 686 is followed for the case where a BG reading submitted between 5 and 15 minutes prior to the end of the hour wait as determined at step 682 is no longer above the lower target as determined at step 683, as it was prior to the 1 hour wait. At this point, it is known that the BG is not in the hypoglycemic range due to step 673. In this case, the system continues providing prompts for BG checks every hour as set at Step 638 in FIG. 6B, and additionally displays a message to the user to check the BG in 1 hour as shown at step 685. At step 686, as the safest course of action, the insulin restart that would have occurred at the end of the hour is abandoned by disabling the timer path 642 of FIG. 6C. The sub-process 670 then proceeds to step 680

The sequence of steps 682, 687, 688 is followed for the case where a BG level is submitted within the first 45 minutes of the hour wait period as determined at step 682. The system simply displays a message that the BG should be rechecked in X minutes, where X is the number of minutes remaining in the hour wait, given by (T+1 hour)−“NOW”, where “NOW” is the present time. At step 688, the prompt for BG checks every hour is overridden and the BG monitor prompt is set to prompt for a BG reading at (T+1 hour)−“NOW” which causes the BG to be checked at an earlier time, namely an hour after Step 640 of FIG. 6B occurred.

Table 2 below illustrates one embodiment of a suitable way to display the glucose levels and insulin administered to a patient who is on a monitor and control process. The columns of Table 2 include the time that a blood glucose measurement was taken, the value of the blood glucose level, the insulin drip rate that was recommended based on the glucose measurement, and the time that the insulin drip rate was changed. One important aspect of this table format is that the glucose level and the related insulin drip rate are presented as a pair, even though they did not occur at exactly the same time. The linked relationship between these two values is an advantageous part of the present glucose monitor and control management system of the present invention since care providers like to quickly see the relationship between the two parameters, and therefore Table 2 emphasizes this relationship by displaying them on the same line. The timestamps of these values are also printed on the line, so that it is clear that they did not necessarily occur at exactly the same time. The separation of the blood glucose measurements and the subsequent change in insulin rate within the monitor and control process is important since such a separation often occurs within the hospital setting. For example, a technician may take and submit a patient's blood glucose level, but a nurse may subsequently change the insulin drip rate. Also, the amount of time that elapses between these two separate events is an important measure of quality of care within the hospital.

TABLE 2

| | | | |
|---|---|---|---|
| | | Insulin | |
| Glucose | Glucose | Drip | Insulin |
| Timestamp | (mg/dL) | (U/hr.) | Timestamp |

| Oct. 6, 2007 10:03 AM | | (Name) Protocol Started, | | | |
|---|---|---|---|---|---|
| | | Target 100-139 mg/dL | | | |
| 10:03 | AM | 122 | 1.5 | 10:05 | AM |
| 10:05 | AM | 1.5 U Insulin Bolus Given | | | |
| 11:09 | AM | 136 | 2.0 | 11:12 | AM |
| 12:37 | PM | 68 | 0.0 | 12:39 | PM |
| 12:44 | PM | ½ AMP D50 Given | | | |
| 1:05 | PM | 77 | 0.0 | 1:05 | PM |
| 1:25 | PM | 102 | 1.0 | 1:27 | PM |
| 2:00 | PM | 116 | 1.0 | | |
| 3:04 | PM | 132 | 2.0 | 3:10 | PM |

| | | |
|---|---|---|
| 3:32 | PM | Protocol Stopped |

Other types of events are also shown in Table 2. For instance, the first line of the table shows when the named protocol was started, and what the target glucose levels are for that protocol. The system of the present invention allows for any number of different protocols to be used, and so the specific protocol chosen for this patient is shown. The third line of the table shows that an insulin bolus of 1.5 U was administered at 10:05 AM. Other types of events that can be shown in this way are alerts, food eaten by the patient, and general notes about the patient entered by the hospital staff among other things.

The monitor and control process and system provides flexibility for the medical staff in responding to protocol recommendations. For example, glucose readings can be entered at any time, since the monitor and control process that implements a protocol bases its decisions on a rate of change of the BG readings. Also, the insulin drip change, which is usually prompted by a glucose reading, is treated as a separate event so that different staff with different levels of medical care authorization can perform these actions, and they can be performed more flexibly at times that are different from the recommendation. While the system allows users to modify or even ignore most of the recommendations, all deviations are recorded and reported. A statistical analysis is also done to report the percentage of deviation from a strict following of the protocol recommendations.

Additional features of the present system allow for comparison and evaluation of the effectiveness of a treatment protocol. For example, data may be extracted from various databases for a plurality of patients in comparable situations or health conditions and following the same protocol and a statistical evaluation and comparison may be done thereby allowing changes to be made to improve one or more patients' medical situation.

Aspects of the system are included to specifically motivate the medical staff to perform the protocol steps on time. For example, escalating alerts are used which may start a few minutes before an action is required and continue until the action is done. All actions require some sort of user confirmation, so that the system is able to determine the action occurred, and when the action occurred. This information may be used in the statistical analysis to provide an evaluation such as “average glucose reading delay” or “average insulin change delay” that may be used to encourage the medical staff to improve if they are slow to respond to recommended actions. A calculation of the average glucose reading delay would first determine the delay for each glucose reading by subtracting the time the reading was due from the time the reading was taken, likely expressing the result in minutes. If the result is <=0 minutes, that indicates the reading was not delayed and a value of 0 would be used in the next calculation. Next, all of the individual delay values would be added together, and the total would be divided by the number of glucose readings to determine the average glucose reading delay. This value could be expressed in any reasonable time unit such as minutes or hours. The average insulin change delay could be calculated in a similar manner.

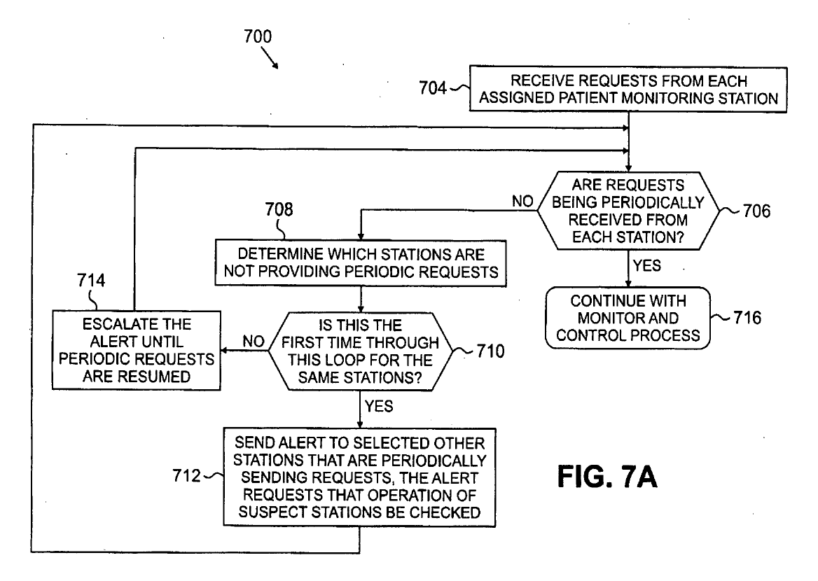

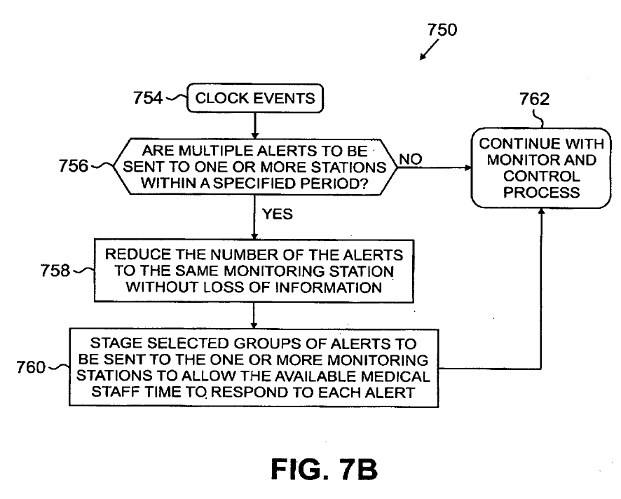

FIG. 7A illustrates a station alert process 700 in accordance with the present invention. At step 704, requests are received from each assigned patient monitoring station. At step 706, it is determined whether the requests are being periodically received from each station. If it is determined that requests are not being periodically received from each station, the process 700 proceeds to step 708. At step 708, the stations that are not providing periodic requests are determined. At step 710, it is determined whether the entry to step 710 is the first time through an alert loop for the same stations. If it is determined that this is the first time through the loop, the process 700 proceeds to step 712. At step 712, an alert is sent to selected other stations that are periodically sending requests, the alert requests that operation of suspect stations be checked. If it is determined that this is not the first time through the loop, the process 700 proceeds to step 714. At step 714, the alert is escalated until periodic requests are resumed. Upon resumption of the periodic requests for a patient monitoring station, the loop controls associated with that patient monitoring station are reset.

Returning to step 706, if it is determined that requests are being periodically received from each station, the process 700 proceeds to step 716 to continue with the monitor and control process.

FIG. 7B illustrates an alert management process 750 in accordance with the present invention. At step 754, clock events, as described above, trigger the alert management process 750. At step 756, it is determined whether there are multiple alerts to be sent to one or more stations within a specified period. If it is determined that there are multiple alerts to be sent to one or more stations within the specified period, then the process 750 proceeds to step 758. At step 758, the number of alerts to the same monitoring station are reduced without loss of information. In this way, the system reduces the burden on medical staff of responding to multiple alerts at the same location within a short time of each other. At step 760, selected groups of alerts to be sent to the one or more monitoring stations are staged to allow the available medical staff time to respond to each alert. The process 750 then proceeds to step 762 to continue with the monitor and control process.

Returning to step 756, if it is determined that multiple alerts are not to be sent to one or more stations within the specified period, the process 750 proceeds to step 762 to continue with the monitor and control process.

While the present invention has been disclosed in a presently preferred context it will be recognized that the present teachings may be adapted to a variety of contexts consistent with this disclosure and the claims that follow. For example, the invention could also be applied to other medical protocols such as heparin drip protocols. It will also be appreciated that variations in the particular hardware and software employed are feasible, and to be expected as both evolve with time. For example, computing devices such as handheld computing devices, IV pumps with internal computing facilities, biological sensors, such as implantable blood glucose sensors and the like are expected to evolve with time and technology developments. New versions of the monitor and control process may incorporate more statistical analysis and prediction techniques to provide a user with the ability to forecast changes in a patients medical status and provide recommendations to appropriately respond to such predictions. Other such modifications and adaptations to suit a particular medical application will be readily apparent to those of ordinary skill in the art.

We claim:

1. A method for adapting management of medical procedures for a patient assigned to a medical protocol, the method comprising:
receiving periodically at a management process a report of a patient's monitored physiological data and confirmed medical procedures from a patient's monitoring station;
adapting an instruction for management of the medical procedures for the patient in response to an evaluation of the patient's periodic reports; and
sending the adapted instruction from the management process to the patient's monitoring station or to selected other monitoring stations based on the evaluation.

2. The method of claim 1 further comprises:
receiving an updated patient report of the patient's monitored physiological data in response to a first medical procedure according to the adapted instruction, wherein the first medical procedure is a member of a first set of medical procedures which can be accomplished by a member of the medical staff having authorization for the first set of medical procedures; and
adapting a second instruction for patient management in response to an evaluation of the updated patient's report, wherein the second adapted instruction includes a request for a second medical procedure based on the medical protocol and a request for confirmation that the second medical procedure has been accomplished, wherein the second medical procedure is a member of a second set of medical procedures.

3. The method of claim 2, further comprising:
sending the second adapted instruction from the management process to the patient's monitoring station; and
confirming the second medical procedure according to the second adapted instruction has been accomplished by the member of the medical staff having authorization for the second set of medical procedures.

4. The method of claim 2 wherein the first set of medical procedures includes taking a blood glucose reading and wherein the second medical procedure includes changing an insulin infusion drip rate.

5. The method of claim 3 further comprises:
sending an alert to the patient's monitoring station indicating the second medical procedure has not been confirmed within a specified period; and
sending repeatedly an escalating the alert to the patient's monitoring station until the confirmation is received.

6. The method of claim 1 further comprises:
sending an alert to the selected other monitoring stations in response to not receiving the patient's periodic reports at the management process, the alert requesting that operation of the patient's monitoring station be checked.

7. The method of claim 1 further comprises:
determining whether multiple alerts are to be sent to one or more monitoring stations within a specified time interval;
reducing the number of alerts sent to the same monitoring station without loss of information; and
staging selected groups of alerts to be sent to the one or more monitoring stations to allow the available medical staff time to respond to each individual alert.

8. The method of claim 1 wherein the evaluation includes an assessment of when a medical procedure was accomplished and selected physiological data to determine whether to adapt a subsequent instruction.

9. The method of claim 8 wherein the subsequent instruction is adapted in response to the elapsed time since the selected physiological data has been checked, the subsequent instruction requesting a medical procedure be accomplished within a time period that is less than or greater than a time recommended for the medical procedure provided by the medical protocol, wherein the variation in the time period from the time recommended is specified based on the selected physiological data.

10. The method of claim 2 wherein the updated patient report indicates that the medical procedure according to the adapted instruction was not followed and a reason for not following the medical procedure is submitted to the management process.

11. The method of claim 1 further comprises:
calculating for each patient an average response time categorized by medical procedures, wherein a response time is measured from the time of sending an instruction requesting a medical procedure to receiving a confirmation that the medical procedure has been accomplished; and
reporting, by category of the medical procedures, each patient's average response times and a group average response time for all patients being managed.

12. A system for monitoring a patient and providing instructions to a medical staff for controlling one or more of a patient's physiological parameters, the system comprising:
a computing device for running a program to monitor a patient and generate instructions to control a physiological parameter of the patient;
a user terminal associated with the patient for monitoring and communicating instructions to the medical staff and for receiving the one or more of the patient's physiological parameters from the medical staff which are then sent to the computing device;
a blood sampling and analysis means for providing an analysis of a blood sample taken from the patients the analysis in a suitable form to be submitted to the computing device; and
a medication administering device adjustable for dosage utilized for controlling one or more doses of medication related to the patient's physiological parameter.

13. The system of claim 12 wherein the computing device is a handheld computer.

14. The system of claim 12 wherein the computing device is an intravenous pump.

15. The system of claim 12 wherein the blood sampling and analysis means is a glucometer for sampling a patient's blood an providing a blood glucose analysis and the medication administering device is an intravenous pump.

16. The system of claim 11 further comprising:
a means for coupling the computing device to the user terminal over the Internet allowing the issuing of instructions and the receiving of patient's reports remote from the patient's monitoring station associated with the patient.

17. A computer-readable medium storing a computer program which causes a computer system to perform a method for adapting management of medical procedures for a patient assigned to a medical protocol, the method comprising:
receiving periodically at a management process a report of a patient's monitored physiological data and confirmed medical procedures from a patient's monitoring station;
adapting an instruction for management of the medical procedures for the patient in response to an evaluation of the patient's periodic reports; and
sending the adapted instruction from the management process to the patient's monitoring station or to selected other monitoring stations based on the evaluation.

18. The computer-readable medium of claim 17 further comprises:
receiving an updated patient report of the patient's monitored physiological data in response to a first medical procedure according to the adapted instruction, wherein the first medical procedure is a member of a first set of medical procedures which can be accomplished by a member of the medical staff having authorization for the first set of medical procedures; and
adapting a second instruction for patient management in response to an evaluation of the updated patient's report, wherein the second adapted instruction includes a request for a second medical procedure based on the medical protocol and a request for confirmation that the second medical procedure has been accomplished, wherein the second medical procedure is a member of a second set of medical procedures.

19. The computer-readable medium of claim 18, further comprising:
sending the second adapted instruction from the management process to the patient's monitoring station; and
confirming the second medical procedure according to the second adapted instruction has been accomplished by the member of the medical staff having authorization for the second set of medical procedures.

20. The computer-readable medium of claim 18 wherein the updated patient report indicates that the medical procedure according to the adapted instruction was not followed and a reason for not following the medical procedure is submitted to the management process.

21. The computer-readable medium of claim 17 further comprises:
determining whether multiple alerts are to be sent to one or more monitoring stations within a specified time interval;
reducing the number of alerts sent to the same monitoring station without loss of information; and
staging selected groups of alerts to be sent to the one or more monitoring stations to allow the available medical staff time to respond to each individual alert.

22. The computer-readable medium of claim 17 wherein the evaluation includes an assessment of elapsed time since selected physiological data has been checked to determine whether to adapt a subsequent instruction to request a medical procedure be accomplished within a time period that is less than or greater than a time recommended for the medical procedure provided by the medical protocol, wherein the variation in the time period from the time recommended is specified based on the selected physiological data.
